(19) United States
(12) Patent Application Publication
(10) Pub. No.: US 2016/0347682 A1
(43) Pub. Date: Dec. 1, 2016

(54) Zeolite Catalyst for Alkyl Halide to Olefin Conversion

(71) Applicant: Sabic Global Technologies, B.V., BERGEN OP ZOOM (NL)

(72) Inventor: Ashim Kumar Ghosh, Houston, TX (US)

(21) Appl. No.: 15/155,686

(22) Filed: May 16, 2016

Related U.S. Application Data
(60) Provisional application No. 62/166,501, filed on May 26, 2015.

(51) Int. Cl. C07C 1/30

(52) U.S. Cl. CPC C07C 1/30; C07C 2529/70

(57) ABSTRACT

A method for converting an alkyl halide to an olefin, the method comprising contacting a crystalline zeolite catalyst having an STI framework topology with a feed comprising the alkyl halide under reaction conditions sufficient to produce an olefin product comprising C₂ to C₅₊ olefins, wherein the crystalline zeolite catalyst has a compositional formula:

M_{y/n}[SiₓQ_{y}O_{2(x+y)}]

where M is a cation; n is the charge of the cation, y/n is the number of cations; x/y is equal to or greater than 5; and Q is aluminum, gallium iron, boron, indium, or mixtures thereof.

### CROSS-REFERENCE TO RELATED APPLICATIONS

The present application is a non-provisional of and claims priority to U.S. Provisional Patent Application No. 62/166,501 filed May 26, 2015 and entitled “Zeolite Catalyst for Alkyl Halide to Olefin Conversion,” which application is incorporated by reference herein in its entirety.

### TECHNICAL FIELD

This disclosure relates to crystalline zeolite catalysts. More particularly the present disclosure relates to the use of crystalline aluminosilicate microporous zeolite catalysts for selective alkyl halide to olefin conversion.

### BACKGROUND

Methane is the main constituent of natural gas and the largest projected available hydrocarbon source of the future. With the advent of efficient processes for the selective halogenation of methane to halomethane particularly to a monohalomethane (e.g., chloromethane) using a mixed metal oxide or a metal supported catalyst, the possibilities for direct utilization of natural gas to obtain higher hydrocarbons began to unfold. The conversion of an alkyl halide to olefins has attracted interest due to the significance of C-C bond construction from a C₁-reactant. Zeolite catalysts such as ZSM-5 and SAPOs (silico-alumino-phosphates) have all facilitated the conversion of alkyl halides to light olefins such as ethylene, propylene, and butene.

One challenge to achieving commercial success with the use of these catalysts is the inability to selectively produce a desired olefin product or a desired olefin product distribution when converting alkyl halides to light olefins.

### SUMMARY

Disclosed herein is a method for converting an alkyl halide to an olefin, the method comprising contacting a crystalline zeolite catalyst having an STI framework topology with a feed comprising the alkyl halide under reaction conditions sufficient to produce an olefin product comprising C₂ to C₅₊ olefins, wherein the crystalline zeolite catalyst has a compositional formula:

M_{y/n}[SiₓQ_{y}O_{2(x+y)}]

where M is a cation; n is the charge of the cation, y/n is the number of cations; x/y is equal to or greater than 5; and Q is aluminum, gallium iron, boron, indium, or mixtures thereof.

Also disclosed herein is a method for converting an alkyl halide to an olefin, the method comprising contacting a crystalline zeolite catalyst with a feed comprising methyl chloride under reaction conditions sufficient to produce an olefin product having C₂ to C₅₊ olefins, wherein the crystalline zeolite catalyst has an STI framework topology and a pore diameter ranging from 4.0 Å to 5.0 Å.

Also disclosed herein is a crystalline zeolite catalyst capable of converting a feed comprising an alkyl halide to an olefin product comprising C₂ to C₅₊ olefins, wherein the crystalline zeolite catalyst has a compositional formula

M_{n/y}[SiₓQ_{y}O_{2(x+y)]}

where M is a cation; n is the charge of the cation, y/n is the number of cations; x/y is equal to or greater than 5; and Q is aluminum, gallium iron, boron, indium, or mixtures thereof; and wherein the olefin product comprises equal to or greater than 50% propylene.

Also disclosed herein is a system for producing olefins, the system comprising an inlet for a feed comprising an alkyl halide; a reaction zone that is configured to be in fluid communication with the inlet; wherein the reaction zone comprises any one of the disclosed crystalline zeolite catalysts; and an outlet configured to be in fluid communication with the reaction zone to remove an olefin product from the reaction zone.

### BRIEF DESCRIPTION OF THE DRAWINGS

FIGS. 1A and 1B provide non-limiting uses of ethylene (FIG. 1A) and propylene (FIG. 1B) produced from the catalysts and processes of the present disclosure.

FIG. 2 illustrates a system which can be used to convert alkyl halides to olefin products with the crystalline zeolite catalyst of the present disclosure.

### DETAILED DESCRIPTION

Disclosed herein are catalysts for the conversion of alkyl halides to olefins and methods of using same. In an embodiment, the catalyst comprises a zeolite and the method comprises the selective conversion of an alkyl halide to a user and/or process desired olefin product or olefin product distribution.

In an embodiment, a catalyst suitable for use in the present disclosure comprises a crystalline zeolite. In an embodiment the crystalline zeolite catalyst may be characterized by

Formula I

M_{y/n}[SiₓAl_{y}O_{2(x+y)]} (I)

where M is a cation, n refers to the charge of the cation, and y/n is the number of cations. Silica and alumina atoms in the framework structure are referred to as T atoms. M can be a mono or divalent cation such as an alkali metal cation; alkaline earth metal cation, or combinations thereof and n is 1 or 2. The zeolite framework may contain gallium (Ga), boron (B), iron (Fe), indium (In), or combinations thereof as substitutions for at least some of the T atoms. The x/y ratio can be equal to or greater than 5, alternatively greater than 20, alternatively greater than 40, or alternatively greater than 100. If a T atom is substituted with another atom, for example Ga, then y in the x/y ratio is inclusive of the substituted atom, that is Si/(Al+Ga).

In an aspect, the crystalline zeolite catalyst is characterized by a STI framework topology of the type depicted as Structure I at the highest possible topological symmetry, Fmmm:

which is characterized by a two-dimensional channel system delineated by 10-membered rings connected through 8-membered rings.

In an embodiment, the crystalline zeolite catalyst as-synthesized is converted to its acidic form. The method for conversion of the crystalline zeolite catalyst to its acidic form may comprise removal of an organic structure directing agent (OSDA), also known as an organic template, by separating and washing the formed zeolite material from the synthesis gel mixture to form a washed zeolite material. The washed zeolite material may then be calcined by heating the material to a temperature of from 400° C. to 600° C., or alternatively from 450° C. to 550° C. for a time period of from 1 hour (h) to 10 hours, or alternatively from 2 h to 5 h to form a heated catalyst. If the as-synthesized zeolite contains no OSDA then a template removal step is not necessary. The heated catalyst may subsequently be subjected to ion-exchange using a source of ammonium ions resulting in an ammonium ion-exchanged zeolite catalyst. The NH₄-exchanged zeolite catalyst may be calcined at temperatures of greater than 400° C., or alternatively from 450° C. to 550° C. for a time period of from 1 h to 10 h, or alternatively from 2 h to 5 h to produce the crystalline zeolite catalyst in its acidic form. In particular instances, the crystalline zeolite catalyst in its acidic form, hereinafter designated CAT-H^{P}, has a total acid concentration of from 0.01 mmole/g-catalyst to 3.0 mmole/g-catalyst, or alternatively from 0.1 mmole/g-catalyst to 1.0 mmole/g-catalyst.

In an aspect the CAT-H⁺ can have an average particle size of 0.2 μm to 0.7 μm, or alternatively from 0.3 μm to 0.5 μm. The crystalline zeolite catalysts of the present disclosure (i.e., CAT-H⁺) can have an average pore opening diameter of from 3.5 Å to 5.5 Å, or alternatively from 4.0 Å to 5.0 Å.

In an embodiment, a crystalline zeolite catalyst suitable for use in the present disclosure is SSZ-75 and may be characterized by Formula II

H_{y}[SiₓAl_{y}O_{2(x+y)}] (II)

where Formula II refers to the acidic form of the crystalline zeolite that may contain monovalent, divalent, or trivalent cations as charge compensating ions. In an embodiment, the ratio of x to y (x/y) is equal to or greater than 5. In an embodiment, the crystalline zeolite catalyst is SSZ-75 and is characterized by an x/y ratio of equal to or greater than 5 where x is the number of silicon (Si) atoms and y is the number of aluminum atoms. The catalyst may contain gallium (Ga), boron (B), iron (Fe), indium (In), or combinations thereof as substitutions for at least a portion of the T atoms present in the zeolite framework. Thus, y is the sum of aluminum and other substituted T atoms. The x/y ratio can be greater than 5, alternatively greater than 20, alternatively greater than 40, or alternatively greater than 100. SSZ-75 and processes of making same are disclosed in U.S. Pat. No. 7,713,512 and U.S. Patent Application Publication No. 2007/0284284 which are incorporated by reference herein in its entirety. SSZ-75 suitable for use in the present disclosure is in its acidic form and may have been converted to same using any suitable methodology, such as those disclosed herein. Consequently, in an embodiment, CAT-H^{P} is SSZ-75.

A method of the present disclosure comprises the conversion of alkyl halides to olefins with a crystalline zeolite catalyst (e.g., SSZ-75). The method may comprise contacting the crystalline zeolite catalyst (e.g., SSZ-75) with a feed comprising an alkyl halide under reaction conditions sufficient to produce an olefin product. In an embodiment, the feed includes one or more alkyl halides. For example, the feed may comprise alkyl mono halides, alkyl dihalides, and alkyl trihalides. Alternatively, the feed comprises alkyl mono halides with less than 10% of other halides (e.g., dihalides, trihalides) relative to the total halides. In another embodiment, the feed comprises less than 10 mole % of a dialkyl halide, alternatively less than 1 mole %. In yet another embodiment, the feed comprises less than 10 mole % of a trialkyl halide or alternatively less than 1 mole %. Alternatively, the feed consists essentially of a mono alkyl halide. The alkyl halide feed may also contain inert diluents such as nitrogen, helium, steam, and the like.

In an embodiment, the feed comprises alkyl halides having the following chemical structure: CₙH₍₂ₙ₊₂₎₋ₘXₘ, where n and m are integers, n ranges from 1 to 5, alternatively from 1 to 3, alternatively 1; m ranges 1 to 3, alternatively 1; and X is Br, F, I, or Cl. Non-limiting examples of methyl halides suitable for use in the present disclosure include methyl chloride, methyl bromide, methyl fluoride, methyl iodide, or combinations thereof. In particular aspects, the feed may include 10, 15, 20, 40, 50 mole % or more of the alkyl halide. In particular embodiments, up to 20 mole % of the feed includes an alkyl halide. In an embodiment, the alkyl halide is methyl chloride. Alternatively, the alkyl halide is methyl chloride or methyl bromide.

In an embodiment, a method of the present disclosure comprises the conversion of alkyl halides to light olefins such as ethylene and propylene using the disclosed crystalline zeolite catalysts (e.g., SSZ-75). In particular, the method comprises contacting an alkyl halide feed of the type disclosed herein with a crystalline zeolite catalyst, also of the type disclosed herein (e.g., SSZ-75) to produce an olefin product having C₂ to C₅₊ olefins. The following non-limiting two-step process is an example of conversion of methane to methyl chloride and conversion of methyl chloride to ethylene and propylene. The second step illustrates the reactions that are believed to occur in the context of the present disclosure

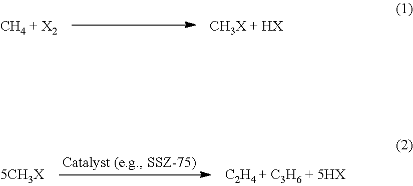

where X is Br, F, I, or Cl. Besides the light olefins the reaction may produce byproducts such as methane, C₄-C₅ olefins, and aromatic compounds such as benzene, toluene. and xylene.

Conditions sufficient for olefin production (e.g., ethylene and propylene as shown in Equation 2) include temperature, time, alkyl halide concentration, space velocity, and pressure. In an embodiment, the temperature for olefin production may range from 300° C. to 500° C., alternatively ranging from 350° C. to 450° C. In another aspect, the temperature range is from 325° C. to 375° C. A weight hour space velocity (WHSV) of higher than 0.5 h⁻¹ can be used, or alternatively between 0.5 and 10 h⁻¹. The conversion of alkyl halide is carried out at a pressure of atmospheric, or alternatively at a pressure less than 100 psig or alternatively less than 20 psig. The conditions for olefin production may be varied based on the type or size of reactor.

In some embodiments, the crystalline zeolite catalyst (SSZ-75) is regenerated after 20, 25, 30, 35, or 40 hours of use in converting the alkyl halide to the olefin product.

In an embodiment, the conversion of a feed comprising a monoalkyl halide to an olefin using a crystalline zeolite catalyst (e.g., SSZ-75) results in an olefin product comprising propylene as the major product.

Catalytic activity as measured by alkyl halide conversion can be expressed as the % moles of the alkyl halide converted with respect to the moles of alkyl halide fed. As an example, methyl chloride (CH₃Cl) is used here to define conversion and selectivity of products by the following formulas:

$$\% {CH}_{3} Cl Conversion = \frac{( {CH}_{3} Cl ) ° - ( {CH}_{3} Cl )}{( {CH}_{3} Cl ) °} \times 100$$

where, (CH₃Cl)⁰ and (CH₃Cl) are moles of methyl chloride in the feed and reaction product, respectively.

Selectivity for propylene may be expressed as:

$$\% Propylene Selectivity = \frac{3 ( {C}_{3} {H}_{6} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) + …\end{matrix}} \times 100$$

where, the numerator is the carbon adjusted mole of propylene and the denominator is the sum of the carbon adjusted moles of all hydrocarbons in the product stream. In some aspects, the crystalline zeolite catalysts (e.g., SSZ-75) show a selectivity to propylene of equal to or greater than 50%, alternatively equal to or greater than 55, 60, 65, or 75% after reaction conditions which include for example a temperature range of between 300 ° C. and 500° C. (e.g., 350° C.) and a pressure less than 20 psig.

Selectivity for ethylene may be expressed as:

$$\% Ethylene Selectivity = \frac{2 ( {C}_{2} {H}_{4} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) +\end{matrix}} \times 100$$

where, the numerator is the carbon adjusted moles of ethylene and the denominator is the sum of all the carbon adjusted mole of all hydrocarbons in the product stream. In some aspects, the crystalline zeolite catalysts (e.g., SSZ-75) show a selectivity to ethylene of equal to or less than 10%, alternatively reaction conditions which include for example a temperature range of between 300 ° C. and 500° C. (e.g., 350° C.) and a pressure less than 20 psig.

In some aspects, the crystalline zeolite catalysts (e.g., SSZ-75) show a selectivity that results in less than 10% of the total amount of olefin product being C₅ and/or C₅₊ olefins, or alternatively less than 5%. In some aspects, the crystalline zeolite catalysts (e.g., SSZ-75) show a selectivity that results in less than 0.1% of the total amount of olefin product being aromatics. In some aspects, the crystalline zeolite catalysts (e.g., SSZ-75) show a selectivity that results in the production of an olefin product having a product distribution of from 70% to 90% for the production of C₂ and C₃ olefins.

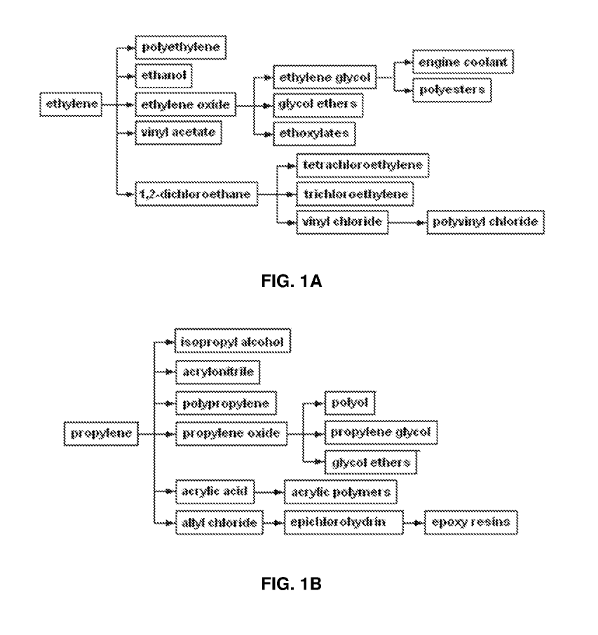

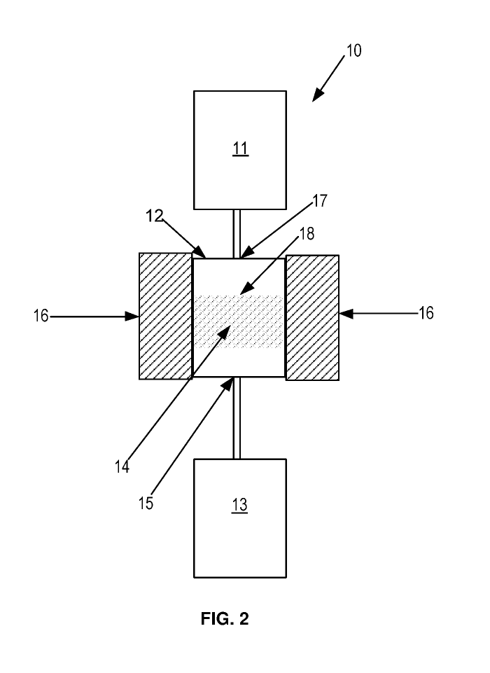

Referring to FIG. 2, a system 10 is illustrated, which can be used to convert alkyl halides to olefin products with the crystalline zeolite catalyst of the present disclosure. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the crystalline zeolite catalyst (e.g., SSZ-75) 14 of the present disclosure. Non-limiting examples of reactors that can be used include fixed-bed reactors, fluidized bed reactors, bubbling bed reactors, slurry reactors, rotating kiln reactors, or any combinations thereof when two or more reactors are used. In some aspects, a fixed bed reactor can be used. The amount of the catalyst 14 used can be modified as desired to achieve a given amount of product produced by the system 10. A non-limiting example of a reactor 12 that can be used is a fixed-bed reactor (e.g., a fixed-bed tubular stainless steel reactor which can be operated at atmospheric pressure). The reactor 12 can include an outlet 15 for products produced in the reaction zone 18. The products produced can include ethylene and propylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., ethylene or propylene) for other uses. By way of example only, FIG. 1 provides non-limiting uses of ethylene (FIG. 1A) and propylene (FIG. 1B) produced from the catalysts and processes of the present disclosure. Still further, the system 10 can also include a heating source 16. The heating source 16 can be configured to heat the reaction zone 18 to a temperature sufficient (e.g., 325° C. to 375° C.) to convert the alkyl halides in the alkyl halide feed to olefin products. A non-limiting example of a heating source 16 can be a temperature controlled furnace. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present disclosure.

The methods of the present disclosure can further include collecting or storing the olefin product along with using the olefin product to produce a petrochemical or a polymer.

The following are enumerated embodiments are provided as non-limiting examples:

A first embodiment which is a method for converting an alkyl halide to an olefin, the method comprising contacting a crystalline zeolite catalyst having an STI framework topology with a feed comprising the alkyl halide under reaction conditions sufficient to produce an olefin product comprising C₂ to C₅₊ olefins, wherein the crystalline zeolite catalyst has a compositional formula: M_{y/n}[SiₓQ_{y}O_{2(x+y)}]where M is a cation; n is the charge of the cation, y/n is the number of cations; x/y is equal to or greater than 5; and Q is aluminum, gallium iron, boron, indium, or mixtures thereof.

A second embodiment which is the method of the first embodiment where M is a monovalent cation, a divalent cation, a trivalent cation, or H.

A third embodiment which is the method of any of the first through second embodiments wherein the crystalline zeolite catalyst comprises SSZ-75.

A fourth embodiment which is the method of any of the first through third embodiments wherein the crystalline zeolite catalyst is in an acidic form.

A fifth embodiment which is the method of the fourth embodiment wherein the acidic form is provided by heating the as-synthesized crystalline zeolite followed by ion-exchange with NH₄⁺ ions and calcining at equal to or greater than 400° C.

A sixth embodiment which is the method of any of the first through fifth embodiments wherein the crystalline zeolite catalyst has a particle size of from 0.2 μm to 0.7 μm.

A seventh embodiment which is the method of any of the first through sixth embodiments wherein the crystalline zeolite catalyst has a pore opening of diameter of 3.5 Å to 5.5 Å.

An eighth embodiment which is the method of any of the first through seventh embodiments wherein the olefin product comprises equal to or greater than 50% propylene.

A ninth embodiment which is the method of any of the first through seventh embodiments wherein the olefin product comprises equal to or greater than 75% propylene.

A tenth embodiment which is the method of any of the first through ninth embodiments wherein the olefin product comprises equal to or less than 5% total amount of C₅ and C₅₊ olefins.

An eleventh embodiment which is the method of any of the first through tenth embodiments wherein the olefin product comprises equal to less than 10% C₂ olefins.

A twelfth embodiment which is the method of any of the first through eleventh embodiments wherein the alkyl halide is an alkyl mono halide.

A thirteenth embodiment which is the method of any of the first through twelfth embodiments wherein the alkyl halide is a methyl halide.

A fourteenth embodiment which is the method of the thirteenth embodiment wherein the methyl halide is methyl chloride, methyl bromide, methyl fluoride, methyl iodide, or any combinations thereof.

A fifteenth embodiment which is the method of the thirteenth embodiment wherein the alkyl halide is methyl chloride.

A sixteenth embodiment which is the method of any of the first through fifteenth embodiments wherein the feed comprises equal to or greater than 10 mole % of the alkyl halide.

A seventeenth embodiment which is the method of any of the first through sixteenth embodiments wherein the feed comprises at least a second alkyl halide (di- and tri-halide methane) in an amount of less than 10 mole % relative to the total halide in the feed.

An eighteenth embodiment which is a method for converting an alkyl halide to an olefin, the method comprising contacting a crystalline zeolite catalyst with a feed comprising methyl chloride under reaction conditions sufficient to produce an olefin product having C₂ to C₅₊ olefins, wherein the crystalline zeolite catalyst has an STI framework topology and a pore diameter ranging from 4.0 Å to 5.0 Å.

A nineteenth embodiment which is the method of the eighteenth embodiment wherein the crystalline zeolite catalyst has a compositional formula M_{y/n}[SiₓQ_{y}O_{2(x+y)}] where M is a cation; n is the charge of the cation, y/n is the number of cations; x/y is equal to or greater than 5; and Q is aluminum, gallium iron, boron, indium, or mixtures thereof.

A twentieth embodiment which is the method of any of the eighteenth through nineteenth embodiments wherein the crystalline zeolite catalyst comprises SSZ-75.

A twenty-first embodiment which is the method of any of the eighteenth through twentieth embodiments wherein the feed comprises equal to or greater than 10 mole % methyl chloride.

A twenty-second embodiment which is the method of any of the eighteenth through twenty-first embodiments wherein an olefin product comprises equal to or greater than 50% propylene.

A twenty-third embodiment which is the method of any of the eighteenth through twenty-second embodiments wherein the olefin product comprises equal to or less than 5% C₅ or C₅₊ olefins.

A twenty-fourth embodiment which is the method of any of the eighteenth through twenty-second embodiments wherein the olefin product comprises equal to less than 10% C₂ olefins.

A twenty-fifth embodiment which is the method of any of the eighteenth through twenty-fourth embodiments wherein an olefin product of C₂ and C₃ olefins is from 70% to 90%.

A twenty-sixth embodiment which is the method of any of the first through twenty-fifth embodiments further comprising using the olefin product to produce a petrochemical or polymer.

A twenty-seventh embodiment which is the method of any of the first through twenty-sixth embodiments further comprising regenerating the crystalline zeolite catalyst after 20, 25, 30, 35, or 40 hours of use in converting the alkyl halide to the olefin.

A twenty-eighth embodiment which is the method of any of the first through twenty-seventh embodiments wherein reaction conditions sufficient to produce the olefin product comprise a temperature of equal to or greater than 300° C., a weight hourly space velocity of equal to or greater than 0.80/h and a pressure of atmospheric.

A twenty-ninth embodiment which is the method of any of the first through twenty-eighth embodiments wherein the aromatics selectivity of the crystalline zeolite catalyst is less than 0.1%

A thirtieth embodiment which is a crystalline zeolite catalyst capable of converting a feed comprising an alkyl halide to an olefin product comprising C₂ to C₅₊ olefins, wherein the crystalline zeolite catalyst has a compositional formula M_{y/n}[SiₓQ_{y}O_{2(x+y)}] where M is a cation; n is the charge of the cation, y/n is the number of cations; x/y is equal to or greater than 5; and Q is aluminum, gallium iron, boron, indium, or mixtures thereof; and wherein the olefin product comprises equal to or greater than 50% propylene.

A thirty-first embodiment which is the crystalline zeolite catalyst of the thirtieth embodiment comprising SSZ-75.

A thirty-second embodiment which is the crystalline zeolite catalyst of any of the thirtieth through thirty-first embodiments wherein the crystalline zeolite catalyst is in an acidic form.

A thirty-third embodiment which is the crystalline zeolite catalyst of any of the thirtieth through thirty-second embodiments wherein the acidic form is provided by heating the as-synthesized zeolite followed by ion-exchange with NH₄⁺ ions and calcining at equal to or greater than 400° C.

A thirty-fourth embodiment which is the crystalline zeolite catalyst of any of the thirtieth through thirty-third embodiments wherein the crystalline zeolite catalyst has a particle size of from 0.2 μm to 0.7 μm.

A thirty-fifth embodiment which is the crystalline zeolite catalyst of any of the thirtieth through thirty-fourth embodiments wherein the crystalline zeolite catalyst has a pore opening of having diameter of 4.0 Å to 5.0 Å.

A thirty-sixth embodiment which is a system for producing olefins, the system comprising: an inlet for a feed comprising an alkyl halide; a reaction zone that is configured to be in fluid communication with the inlet; wherein the reaction zone comprises any one of the crystalline zeolite catalysts of any of the thirtieth through thirty-fifth embodiments; and an outlet configured to be in fluid communication with the reaction zone to remove an olefin product from the reaction zone.

A thirty-seventh embodiment which is the system of the thirty-sixth embodiment, wherein the reaction zone further comprises the feed and the olefin product.

A thirty-eighth embodiment which is the system of any of the thirty-sixth through thirty-seventh embodiments, wherein the olefin product comprises ethylene and propylene.

A thirty-ninth embodiment which is the system of any of the thirty-sixth through thirty-eighth embodiments, further comprising a collection device that is capable of collecting the olefin product.

While embodiments of the present disclosure have been shown and described, modifications thereof can be made by one skilled in the art without departing from the spirit and teachings of the disclosure. The embodiments described herein are exemplary only, and are not intended to be limiting. Many variations and modifications of the disclosure are possible and are within the scope of the invention. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Background is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed it is intended to disclose or claim individually each possible number that such a range could reasonably encompass, including any sub-ranges encompassed therein. When describing a range of measurements every possible number that such a range could reasonably encompass can, for example, refer to values within the range with one significant digit more than is present in the end points of a range. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure.

What is claimed is:

1. A method for converting an alkyl halide to an olefin, the method comprising contacting a crystalline zeolite catalyst having an STI framework topology with a feed comprising the alkyl halide under reaction conditions sufficient to produce an olefin product comprising C2 to C5+ olefins,
wherein the crystalline zeolite catalyst has a compositional formula:
M_{y/n}[SiₓQ_{y}O_{2(x+y)}]
where M is a cation;
n is the charge of the cation, y/n is the number of cations;
x/y is equal to or greater than 5; and
Q is aluminum, gallium iron, boron, indium, or mixtures thereof.

2. The method of claim 1 wherein the alkyl halide is a methyl halide.

3. The method of claim 1 wherein the alkyl halide is methyl chloride.

4. The method of claim 1 wherein the crystalline zeolite has a pore diameter ranging from 3.5 Å to 5.5 Å.

5. A method for converting an alkyl halide to an olefin, the method comprising contacting a crystalline zeolite catalyst with a feed comprising methyl chloride under reaction conditions sufficient to produce an olefin product having C₂ to C₅₊olefins, wherein the crystalline zeolite catalyst has an STI framework topology and a pore diameter ranging from 4.0 Å to 5.0 Å.

6. The method of claim 5 wherein the crystalline zeolite catalyst has a compositional formula
M_{y/n}[SiₓQ_{y}O_{2(x+y)}]
where M is a cation;
n is the charge of the cation, y/n is the number of cations;
x/y is equal to or greater than 5; and
Q is aluminum, gallium iron, boron, indium, or mixtures thereof.

7. The method of claim 1 where M is a monovalent cation, a divalent cation, a trivalent cation, or H.

8. The method of claim 1 wherein the crystalline zeolite catalyst comprises SSZ-75.

9. The method of claim 8 wherein the crystalline zeolite catalyst is in an acidic form.

10. The method of claim 9 wherein the acidic form is provided by heating the as-synthesized crystalline zeolite followed by ion-exchange with NH₄⁺ions and calcining at equal to or greater than 400° C.

11. The method of claim 4 wherein the crystalline zeolite catalyst has a particle size of from 0.2 μm to 0.7 μm.

12. The method of claim 1 wherein the olefin product comprises equal to or greater than 50% propylene, preferably equal to or greater than 75% propylene.

13. The method of claim 1 wherein the olefin product comprises equal to or less than 5% total amount of C₅ and C₅₊olefins.

14. The method of claim 1 wherein the olefin product comprises equal to less than 10% C₂ olefins.

15. The method of claim 1 wherein the feed comprises equal to or greater than 10 mole % of the alkyl halide.

16. The method of claim 1 wherein the feed comprises at least a second alkyl halide (di- and tri-halide methane) in an amount less than 10 mole % relative to the total halide in the feed.

17. The method of claim 5 wherein the feed comprises equal to or greater than 10 mole % methyl chloride.

18. The method of claim 1 wherein the olefin product comprises from about 70% to about 90% C₂ and C₃ olefins.

19. The method of claim 1 further comprising regenerating the crystalline zeolite catalyst after 20, 25, 30, 35, or 40 hours of use in converting the alkyl halide to the olefin.

20. The method of claim 1 wherein the aromatics selectivity of the crystalline zeolite catalyst is less than 0.1%.
